(19) United States
(12) Patent Application Publication
(10) Pub. No.: US 2006/0141469 A1
(43) Pub. Date: Jun. 29, 2006

(54) Multi-layered electrochemical microfluidic sensor comprising reagent on porous layer

(75) Inventors: Joel Stephane Rossier, Vionnaz (CH); Frederic Reymond, La Conversion (CH); Patrick Morier, Blonay (CH)

Correspondence Address: HOWSON AND HOWSON, SUITE 210, 501 OFFICE CENTER DRIVE, FT WASHINGTON, PA, 19034, US

(73) Assignee: DiannoSwiss S.S., Monthey (CH)

(21) Appl. No.: 10/543,762

(22) PCT Filed: Jan. 14, 2004
(86) PCT No.: PCT/EP04/01013

§ 371 Date: Jul. 13, 2005

(30) Foreign Application Priority Data
Jan. 14, 2003 (GB) 03008208

(51) Int. Cl. C12Q 1/68; C12M 1/34; G01N 33/558

(52) U.S. Cl. 435/6; 436/514; 435/287.2

(57) ABSTRACT

The present invention relates to a microfluidic electrochemical sensor apparatus and a method for conducting analytical tests with said apparatus for multi-reactant assays. The apparatus of this invention is a multi-layer body made of at least three layers, the first one being a polymer layer (1) comprising a microstructure (5) with at least one integrated microelectrode (4) and conductive tracks (13) for connection to an external electrochemical unit, the second one being a non-porous material serving to cover said microstructure so as to enable microfluidic manipulations and the third one being a porous layer (2) such as a membrane or a glass frit, said porous layer comprising at least one reagent (3) to be solubilized upon contact with a test solution (7) and reacting with an analyte (6) present in said solution to form a product that is transported along said microstructure so as to enable electrochemical detection of said analyte. The invention notably enables the performance of multi-reactant assays in a reduced number of steps.

### BACKGROUND TO THE INVENTION

Many qualitative or semi-quantitative chemical or biochemical assays are performed with a solid support, which is often a porous layer, where a reagent is stored dried and reacts when the solution to be tested wets this solid support or when the solid support is immersed or placed in contact with said test solution Well-known examples of such assay apparatuses are the strips used to determine the pH of a solution or those used to diagnose the presence of a given analyte by immunological or enzymatic assays (as e.g. in pregnancy tests or, respectively, glucose monitoring).

In the first case, the pH is measured by immersion of a strip in an aqueous solution, where some pH indicators are dissolved and change their color depending on the pH of the solution. This system is very convenient because it rapidly gives a first estimation of the actual pH of the solution. Nevertheless, depending on the experimenter or on the daylight, the perception of the color may slightly change, and the measurement cannot be taken as quantitative as the one of the pH meter with pH electrode. Such strips are thus used for semi-quantitative assessment of the pH of a solution.

In other cases, reagents dried on a porous layer are very popular for the measurement of health markers or of special states such as pregnancy or ovulation. Many different designs of such strips have already been disclosed (U.S. Pat. No. 6,399,398; EP025863; EP0456308; U.S. Pat. No. 5,786,220; EP1003037), and they are all based on the following assay principle, which is generally referred to as immuno-chromatography: A porous membrane is used as a porous layer with reagents immobilized or dried in different sections of the strip, the reagents being antibodies marked with latex particles or gold colloids that will be dissolved by a test solution containing an antigen (the analyte or compound to be tested), said antigen binding said marked antibody, thereby forming a complex; this complex is then transferred by capillary flow to a secondary antibody section, in which these secondary antibodies are immobilized and capture the complex. As a result, a colored band appears on the porous layer at the secondary antibody section only if the antigen is present in the solution. This method is mainly used for qualitative assays because the interpretation of the band intensity and its correlation with the antigen concentration is very difficult. In most analytical systems, the detection is achieved directly within the porous layer which renders a washing step difficult or impossible prior to the detection.

We presented earlier some methods of performing quantitative assays in Microsystems (mainly fluidic microchannels) with integrated electrochemical detector or with fluorescence detection (J. S. Rossier and H. H. Girault, Lab Chip, 1, 2001, 153-157; J. S. Rossier, F. Reymond and P. E. Michel, Electrophoresis, 2002, 23, 858-867; J. S. Rossier, C. Vollet, M. Martinelli, A. Carnal, G. Lagger, V. Gobry, P. Michel, F. Reymond and H. H. Girault, Lab Chip, 2002, 2, 145-150) where multi-step reactions were performed by sequential addition of different reagents. For example, immunoassays were performed by immobilising antibodies on the surface of the covered microchannel, and, after blocking with bovine serum albumin (BSA), the chip was ready for a test. Here, the test solution comprising the antigen to be measured is first introduced into the covered microchannel, and the target antigens are specifically captured by the immobilised antibodies to form antigen-antibody complexes. After a washing step, a secondary antibody labelled with an enzyme is introduced inside the covered microchannel and captured on the first antibody-analyte complex. After another blocking step, a solution of substrate is introduced, and an enzymatic reaction starts so as to transform the substrate into a product that can be detected e.g. by electrochemical means, using electrodes present on one wall of the covered microchannel.

The performance of this system is satisfactory but the delivery of different successive reagents implies quite a large infrastructure around the microsystem, as well as cumbersome manipulations which take time and reduce the assay reproducibility. Large automated systems may be used to perform these multiple steps and to control the dispensing of the various reagents. There is a high demand for apparatuses enabling multi-reagent assays to be performed in a reduced number of steps, by unskilled personnel and on portable systems, depending on the applications and fields of use. The present invention discloses an apparatus which meets such requirements, as well as methods of performing such multi-reagent tests with this apparatus.

### PRIOR ART METHODS

As mentioned above, there is a need for a strip composed of a porous layer containing the reagent and followed by a measurement cell such as to give a quantitative answer for the reaction that occurred between a solution and a dried reagent Such a strip exists with applications in enzyme assays such as glucose tests (U.S. Pat. No. 6,241,862, WO0173124), where the electrochemical analysis is made using a membrane containing reagents in the top of a screen-printed electrode such as to provide the reagent directly on the test strip but also to avoid the interferences of the hematocrit level by making a protection of the electroactive surface.

Membranes placed close to a sensor are also used in another type of device that has been proposed by Scheller (DE4216980). In this example, a semi-permeable membrane with reagent is directly placed in contact with an electrochemical transducer. This enables to have a reaction and a detection on the same device. In the present invention, the porous layer containing a reagent is not placed in contact with the sensor portion of the apparatus, but these two parts (porous layer and sensor portion) are separated by a fluidic connection (generally a microstructure such as a microchannel) which allows transfer of the reagent and analyte to the detection portion of the apparatus. In some embodiments of this invention, the detection portion is integrated within this fluidic connection which may also comprise a second reagent (preferably immobilized on and/or dried onto the walls of this fluidic connection). This fluidic connection may also be used to introduce washing steps after the reaction. For example, for removing non-bound conjugate, the presence of the membrane directly on the electrochemical transducer implies that it is impossible to separate the undesired material present in the membrane from the target analyte to detect.

In another disclosure (WO9414066), the authors propose to place a membrane on an electrode so as to enable small analytes to reach the electrode surface by passing through the membrane whereas the antibody-analyte complexes are not liable to cross it and hence to come in direct contact with the electrode. In our invention, the membrane is used to host a reagent that has to permeate the membrane and cross it before being transferred through the fluidic connection to the detection portion of the apparatus.

In another device presenting a membrane on the top of an electrode (FR2692357), the goal is to concentrate detectable positively charged species in a negatively charged layer.

In WO02090573, a permeation layer is present at a defined distance above the electrodes, where some affinity molecules can be present and where an electrochemical analysis can be performed. This arrangement does not allow delivery of a reagent previously present in the permeation layer, and, in addition, it does not allow a controlled fluidic transport towards the detection region as is the case in our system with the fluidic connection This known system is used for preconcentration of the analyte but not for multi-step reactions.

Various known devices combine a membrane with a polymer support for the detection of analytes in a semi-quantitative manner, either by eye or with a reader. In GB 2345133A, the authors place a solid support in contact with a membrane which contains a reagent, and the device enables detection of the presence of an analyte. In this case, there is no microfluidic means (microchannel or the like) that enables further manipulation of the sample for further separation or amplification. In U.S. Pat. No. 5338513, an apparatus is described with a pre-reaction layer and a conjugate layer with a liquid transport layer connecting both preceding layers. The connection layer is made of an absorbent material that is used for the transport from one to the other layer but that cannot be emptied or washed as easily as a microchannel which enables this kind of microfluidic manipulation of the reacted species, the pumping of a fluid via external means or the achievement of washing steps or multi-step assays. In U.S. Pat. No. 5451350, a device is presented where different chambers filled with a porous material are connected through absorbent material. The solution is prevented from passing from one chamber to the other by microfluidic manipulations; it can only be transported through by an absorbent connector that again cannot be rinsed, washed or emptied, which, in contrast, would be the case with a microchannel. In EP 0239174, a substrate is structured with microgrooves in direct contact with a filter in order to transfer filtered biological samples towards an assaying portion of the device. This system is also a transport system that cannot support microfluidic manipulations such as washing or concentrating the sample. In EP0974840 A2, a bladder is fabricated to collect the sample which is further transported towards a detection region. Here again, no means to perform microfluidics are described and the reaction occurring is a clotting reaction, avoiding any washing or further transfer of the reacted sample. WO 00/62060 discloses a system where the molecules are placed in contact with a so-called lateral hybrid device and are transported laterally through a porous layer that again does not contain microfluidic features enabling further microfluidic manipulations. In GB 2322192 A, the device is composed of different materials, one of them being a porous liquid conductive material enabling transfer of the solution towards a zone with a labeled reagent and dragging of the solution on a bed of antibodies immobilized in the porous material such as to develop a band specifically revealing the capture of the antigen. This disclosure does not comprise any microstructure enabling microfluidic manipulations of the reacted sample molecule. In another application (WO0042430A1), a capillary structure is used to deliver the sample to different widdng members for testing different analytes in each of the different wicking members. This system enables to advantageously use microfluidic manipulation in order to distribute the sample into different reactors supported by a porous membrane.

On the other hand, many sensors have been fabricated in solid supports, as described hereinafter, but these solid supports do not comprise porous material for the intake of the sample: WO 93/22053 for example describes a meso-scale device which enables the detection of a sample by means of different reactors that contain an immobilized reagent; this document does not present the coupling with a porous layer that may release a reagent inside the sample solution. WO 99/35497 presents a capillary device filled with a reagent that promotes agglutination of solution inside capillaries; the detection is then performed by the lack of fluidics inside the capillary, which of course prevents any microfluidic manipulations of the sample after reaction.

In analytical tests and especially in microfluidic assay systems, the detection signal generally depends strongly on the hydrodynamics and on the geometrical characteristics of the reactor. In electrochemical sensors, the signal further depends on the electrode size and shape as well as on the diffusion at this electrode. Due to a well-defined microstructure shape and a controlled electrode size and location, the present invention provides a microfluidic electrochemical sensor which is particularly adapted for quantitative and highly sensitive assays. The combination of microfluidics, electrochemical means and integrated reagent in a single apparatus according to the present invention with a porous layer in which the integration of a compound liable to react with an analyte allows the user to reduce the number of steps of the entire assay and to minimize manipulations, external intervention and use of sophisticated instrumentation, whilst maintaining easy and efficient washing as well as substrate incubation or controlled microfluidics and detection.

### DESCRIPTION OF THE INVENTION

The present invention provides a microfluidic assay apparatus according to claim 1 or 2 and a method of performing a microfluidic assay according to claim 13. Preferred or optional features of the invention are defined in the dependent claims.

The apparatus of this invention (hereinafter also referred to as a “test strip”) is a multi-layer body composed of at least three material layers, namely:
(a) a first, polymer layer having at least one fluidic connection comprising at least one microstructure (generally a microchannel or network of microchannels) which possesses at least one inlet and one outlet and which contains at least one microelectrode integrated in said microstructure at a given place and location to form at least one detection portion, said at least one microelectrode being connected, through electrically conductive tracks in contact with said first polymer layer, to an external electrochemical unit (such as a potentiostat, a power supply, an impedance measurement apparatus, etc.);
(b) a second, non-porous layer such as a lamination layer serving to cover said microstructure such as to form a sealed microstructure enabling microfluidic manipulations of the solution through said microstructure;
(c) a third, porous layer comprising or made of a porous material such as a membrane, placed in contact with said first polymer layer at the inlet and/or outlet of said microstructure, said third porous layer comprising at least one dried reagent, said dried reagent being solubilized upon contact with a test solution and reacting with an analyte present in said test solution to form a product (such as an antigen-antibody complex) that is further transported inside said microfluidic microstructure, so as to enable the electrochemical detection of an analyte present in said test solution by way of said at least one integrated microelectrode.

In another embodiment, the reagent comprised in the porous layer of the apparatus of this invention can be irreversibly immobilized in this porous layer, so as to capture either a part of an analyte present in a test solution or an non-desirable compound present in this test solution, so that only the analyte in excess or, respectively, the purified test solution, is transported inside the microstructure to enable detection of either the analyte in excess or, respectively, of an analyte present in the purified test solution by way of the integrated electrode.

In a further embodiment, an analyte may be reversibly immobilized in the porous layer of the apparatus of this invention before reacting with a reagent present in a solution to form a product that is further transported inside the microstructure so as to enable detection of said analyte by way of the integrated electrode(s).

The first polymer layer of the apparatus of this invention can be made of any polymer material, and the microstructure can be fabricated by any method conventionally used for microfabrication such as but not limited to plasma etching, laser photoablation, injection molding, embossing, UV LIGA, polymer casting or silicone technology.

In the present invention, the second non-porous layer can be made of any material enabling waterproof sealing of the microstructure; a polymer or glass can for instance advantageously be used for this purpose. In order to fabricate the apparatus of this invention, this second non-porous layer may for instance be stacked, bonded, pressed, glued or laminated onto the first polymer layer comprising the microstructure to cover in order to enable microfluidic manipulations. A polymer foil (made of e.g. polyethylene, polyethyleneterephthalate, polycarbonate, polystyrene, polyimide or the like) can be laminated under temperature and pressure over the first polymer layer in order to cover the microstructure.

In one embodiment of the present invention, the inlet of the microstructure is formed in the first, polymer layer of the apparatus, whereas the outlet of this microfluidic structure is formed in the second, non-porous layer (for instance by fabrication of an access hole in this second layer). Alternatively, the outlet of the microstructure may be formed in the first, polymer layer and the inlet of the microstructure in the second, non-porous layer. In addition, the microstructure can comprise a plurality of inlets and/or outlets.

In another embodiment, the second non-porous layer may also comprise a microstructure, an electrode and/or electrically conductive tracks.

The microelectrode integrated in the first polymer layer as well as the electrically conductive tracks are may be made of a metal or of a conductive ink. They may also be made of a plurality of materials as for example copper coated with another metal such as gold, silver or platinum or, as another example, silver coated by a salt such as silver chloride. Otherwise, the microelectrode integrated in the first polymer layer in order to be in contact with the solution present in the microstructure may advantageously be placed on the external side of this polymer layer, opposite to the microstructure and be opened be eliminating the polymer material separating the electrically conductive material from the microstructure, thereby creating a recessed electrode, which may for instance be carried out using plasma etching or photoablation. In another embodiment of this invention, this first polymer layer may be formed by two different polymer bodies that are stacked and/or sealed together, a first polymer body containing only the desired microstructure with its inlet and outlet and a microhole for exposition of the microelectrode, and a second polymer body comprising only the electrically conductive tracks and access holes for connection to the inlet and outlet of the microstructure, both bodies being stacked and/or sealed together in such a manner that a portion of the conductive tracks of the second body is placed in connection with the microhole of the first body so as to form a recessed electrode exposed to the microstructure.

For ease of interpretation, in this specification, the term “analyte” refers to any compound to be analyzed using the apparatus of this invention. Generally, the analyte is a molecule which is partially or completely dissolved in a test solution, and it is able to influence the detection after having reacted with a reagent present in the porous layer. In some embodiments, the analyte is directly detected after reaction with the reagent placed in the porous layer. In another embodiment, the analyte is detected indirectly as e.g. in conventional enzymatic or immunological assays where a mediator and/or substrate generate(s) a product that is detected to directly or indirectly assess the number and/or the concentration of the analyte. The analyte may comprise protons, metal ions, small metabolites, kinases, antibodies, antigens, oligonucleotides, DNA, RNA, peptides, proteins or haptamers as well as any other chemical, biological or biochemical compound of interest.

In this specification, the term “reagent” refers to a molecule or a cocktail of different molecules being able to react or to induce a specific reaction or reaction cascade with a species, preferably an analyte, present in the test solution. The reagent may be immobilized, dried, placed as a powder, lyophilized, or placed in a wet medium supported by the porous layer.

In the present specification, the term “test solution” (hereinafter also referred to as “sample solution”) refers to any solution comprising the analyte to be tested or the reagent. In some embodiments, application of the test solution to the apparatus of this invention allows dissolution of the reagent placed in the porous layer, thereby enabling a reaction to take place between the analyte and said reagent. In one embodiment, the analyte to be tested and the reagent placed on the porous layer are transposed, so that the analyte is placed on the porous layer while the test solution only contains one or a plurality of reagent(s).

In this specification, the “porous layer” is a means serving to support a reagent, said porous layer being placed in such a way that the test solution passing through, above or around this porous layer will be able to reach the detection portion by flowing along or across a fluidic connection. The porous layer may be a membrane (e.g. a PVDF or cellulose-based membrane), a fritted glass, a capillary or a microchannel, a bead, a bead bed, a monolithic column, or the like. In one embodiment, more than one porous layer is used to perform multiple reactions either in parallel or in cascade with the same test solution. In some embodiment, a plurality of porous layers may be placed one over the other in order to perform a cascade of reactions. In another embodiment, more than one porous layer is used to support different reagents, one serving to react directly with the analyte (as e.g. to form an antigen-antibody complex) and at least a second one serving to introduce another reagent (as e.g. an enzymatic substrate) at the detection portion of the apparatus by application of a solution (as e.g. water or a buffer solution). In this configuration, the test solution generally passes through only one porous layer, another solution being used to dissolve the other reagent(s) that is(are) requested to perform the reaction cascade. In some embodiments, the porous layer is a membrane or an assembly of membranes enabling the separation of plasma from blood cells and further enabling reaction of a target analyte in this plasma with the reagent(s).

In this specification, the term “fluidic connection” refers to a means enabling to transfer the test solution from the porous layer to the detection portion of the apparatus by laminar flow. In one embodiment, the fluidic connection is a microstructure or a cavity having well-defined geometry and produced in a polymer material. In one embodiment of the invention, the microstructure is a covered microchannel or a network of covered microchannels having at least one inlet and one outlet for uptake and/or withdrawal of fluid. In another embodiment of this invention, the microstructure is a microchannel (or network of microchannels) having al least one dimension of its cross-section smaller than 1 mm so as to maintain a laminar flow of solution along said microstructure and hence enable microfluidic manipulations. When the fluidic connection is composed of a covered microchannel or a cavity it is also possible to force the test solution or another liquid to flow by applying a pressure or by aspirating, such as to displace the test solution or other liquid and/or to wash the fluidic connection and/or to provide a further reagent to the detection portion of the apparatus. In some embodiments, the fluidic connection may also comprise a medium, such as a solid, a gel, a sol-gel, a porous membrane, a monolithic column, beads or packed beads so as to perform additional separation and/or reaction. A reagent may be dried or immobilized (e.g. by physisortpion or covalent binding) on or in this medium, so as to enable further separation or reaction. In another embodiment, the apparatus may comprise more than one fluidic connections so as to provide a plurality of reagents to the detection portion, sequentially or in parallel. In another embodiment, the fluidic connection may comprise a reagent (as e.g. an antibody, an antigen, an enzyme, an oligonudeotide, DNA, RNA, a peptide, a protein, a cell, a ligand, a receptor or the like) which may be dried on the walls of the fluidic connection or immobilized on the walls of the fluidic connection (by physisorption, covalent binding or the like) or in a supporting means like a gel, a sol-gel, beads or the like placed in at least one portion of the fluidic connection. In another embodiment, the fluidic connection serves to connect the apparatus to an external means such as a pump or a separation or detection equipment In another embodiment, the fluidic connection directly comprises a pumping means such as electrodes enabling the generation of electroosmosis.

Herein, the term “detection portion” refers to a means for transducing quantitatively or qualitatively a concentration or the presence of a molecule into a comprehensible signal. In the present invention, this detection portion comprises at least one electrically conductive means (namely one or a plurality of electrodes) that is(are) directly integrated in the fluidic connection with a precise shape and location and that is(are) adapted to perform electrochemical measurements or to electrochemically induce a reaction enabling detection of the compound of interest. In this manner, the electrode(s) allow(s) the user to perform electron or ion transfer reactions (e.g. voltammetric or amperometric measurements), or to electrochemically generate a product detectable by another means like an optical detection system as it is for example the case in electrochemi-luminescence, or to form a spray of solution for further detection by mass spectrometer as it is the case in electrospray ionization mass spectrometry. In one embodiment of this invention, the detection portion can thus comprise, in addition to the electrically conductive means, a means allowing optical detection of a compound like e.g. an optically transparent window or a waveguide. In another embodiment, the detection portion may comprise immobilized molecules enabling a further reaction to take place.

In the present invention, the detection portion is directly integrated in the fluidic connection. For example, one or a plurality of electrodes (and preferably microelectrode(s)) may be integrated in a covered microchannel in the form of a conductive portion. Generally, these electrodes are connected to an external source of electrochemical power by way of electrically conducting tracks in contact with the polymer layer supporting the fluidic connection, thereby enabling for example the measurement of an electrochemical signal which is related to the concentration of an analyte present in the test solution. For certain applications, an array of electrodes may advantageously serve as detection means in order to increase the detection signal.

In another embodiment, the electrode or electrode array may be covered by a protection means preventing adsorption, fouling and/or reaction of undesired species. A membrane or a glass frit may for instance be used for this purpose.

In the present device, the sample solution crosses a porous layer prior to being pumped into a microchannel or other microstructure where the sample can be treated, cleaned, adsorbed and separated from the rest of the reagent due to further microfluidic steps taking place inside the microchannel. This is fundamentally different from systems where both the reaction and the detection are performed inside the porous layer, such as known pregnancy testing systems. The present invention enables detection of low abundant proteins for example hormones such as TSH in low concentration because the background of the non-reacted analyte can be removed after the reacted sample has been transferred from the porous layer inside the microchannel.

### BRIEF DESCRIPTION OF THE DRAWINGS

The invention is further described hereinafter, by way of example only, with reference to the accompanying drawings, in which:

FIG. 1 is a top view of an example of a test strip according to the present invention;

FIG. 2 is a side view of the test strip of FIG. 1;

FIG. 3 is a side view of the test strip showing the reagent placed in the porous layer;

FIG. 4 is a side view of the test strip showing the test solution containing the analyte to be measured;

FIG. 5 is a side view of the test strip showing the analyte reacting with the reagent in the porous layer to form a product;

FIG. 6 is a side view of the test strip showing the analyte-reagent and excess reagent being driven through the fluidic connection to the detection portion;

FIG. 7 is a side view of a test strip according to the invention used for immunological detection of an analyte;

FIG. 8 is a side view of a test strip similar to that shown in FIG. 7 after deposition of the test solution;

FIG. 9 is a side view of the test strip of FIG. 8 after transfer of the conjugate and analyte into the fluidic connection and the detection portion;

FIG. 10 is a side view of the test strip of FIG. 9 after washing of the fluidic connection and dispensing of a substrate molecule;

FIG. 11 is a side view of a test strip similar to that of FIG. 10, in which the substrate molecule is placed in a second porous layer;

FIG. 12 is a side view of the test strip of FIG. 11 after dissolution and dispensing of the substrate inside the detection portion;

FIG. 13 is a side view of a test strip similar to that of FIG. 12, in which the access holes of the fluidic connection are placed one on the top and one at the bottom side of the test strip;

FIG. 14 is a side view of a test strip with more than two access holes;

FIG. 15 is a top view of a test strip with a fluidic connection comprising a covered microchannel network;

FIG. 16 is a view of a test strip and reading equipment according to the invention;

FIG. 17 is a view of the test strip of FIG. 16 placed in the reading equipment of FIG. 16;

FIG. 18 is a view of a syringe modified with a test strip on the edge of said syringe;

FIG. 19 shows the syringe of FIG. 18 with a sample taken in and placed in contact with the test strip for the detection;

FIG. 20 is a view of the modified syringe connected to a reading device;

FIG. 21 shows the results of the detection of aminophenol at different pH values;

FIG. 22 shows the relationship of the detected oxidation potential of aminophenol as a function of the pH corresponding to the experimental results shown in FIG. 21; and

FIG. 23 shows the results of the detection of an enzymatic reaction using the device described in FIGS. 1 to 6.

### DETAILED DESCRIPTION OF PARTICULAR EMBODIMENTS

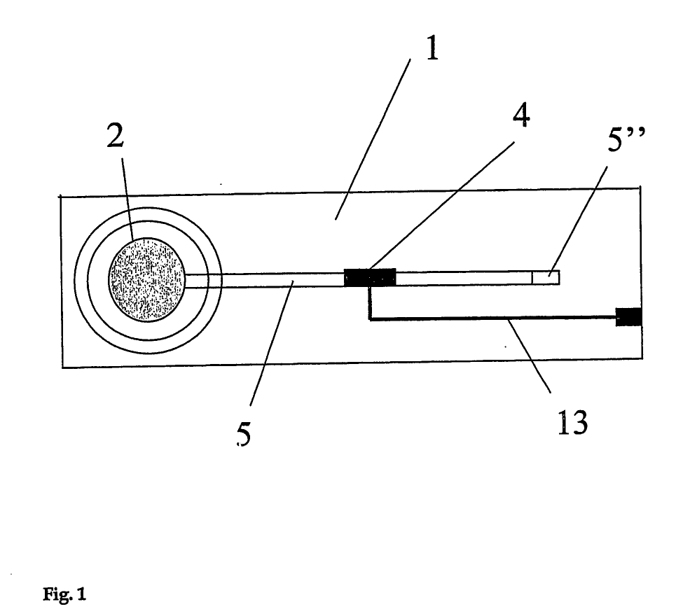

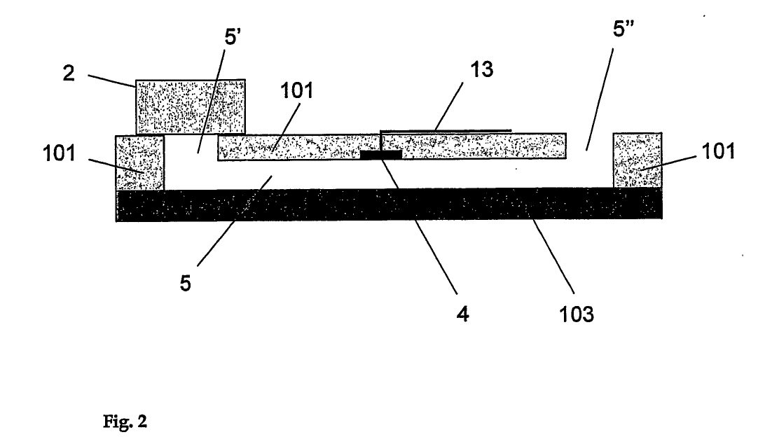

FIGS. 1 and 2 show a test strip which is composed of a covered microchannel 5 formed in a polymer layer 101, with one inlet 5′ and one outlet 5″ enabling microfluidic connection, a second, non-porous layer 103 serving to seal the microchannel, a third porous layer 2, and a detection portion 4 integrated in said microchannel and connected to an external electrochemical workstation by way of electrically conductive tracks 13. In general, the porous layer 2 comprises a membrane stacked on the first polymer layer, which comprises various different access holes and microchannel(s), said microchannel(s) serving in this example as a fluidic connection integrating the detection portion of the test strip.

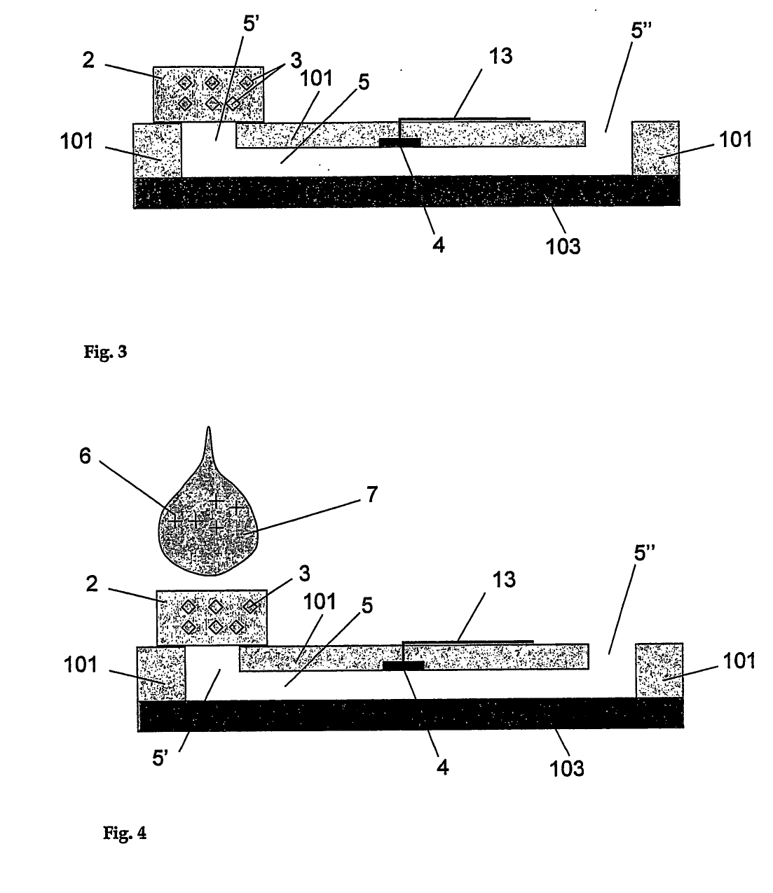

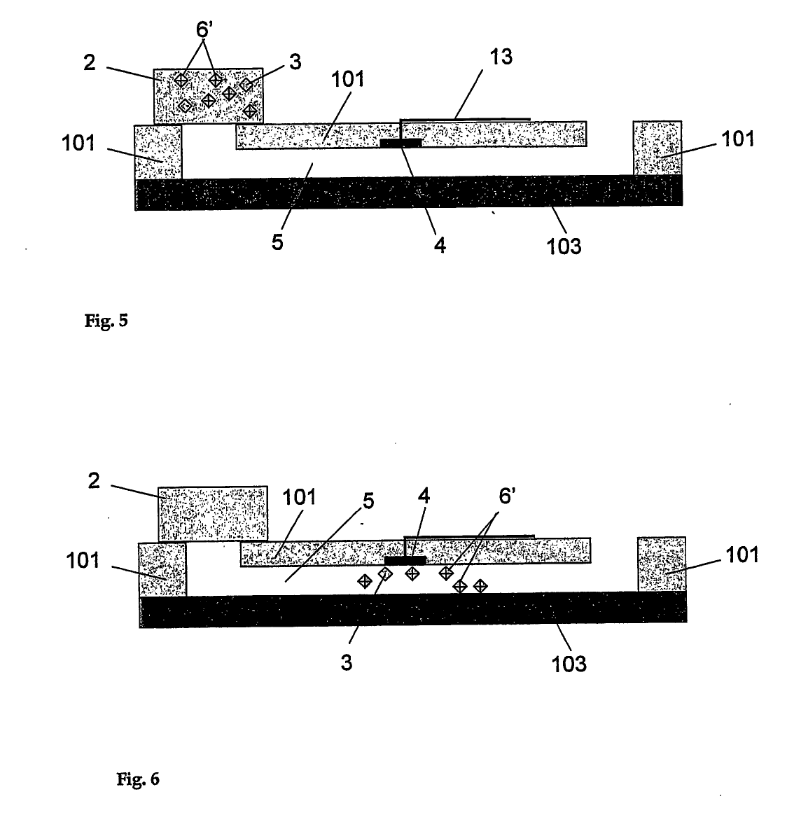

As shown in FIG. 3, a reagent 3 is immobilizled in the porous layer 2. As shown in FIG. 4, a test solution 7 containing an analyte 6 is then introduced through the porous layer 2. The analyte reacts with the reagent 3 to form a reagent-analyte product 6′, as shown in FIG. 5. As shown in FIG. 6, the analyte-reagent and excess reagent are driven through the fluidic connection to the detection portion. The driving force can be capillary flow, electrically driven flow or pumping induced by centrifugation, pressure, aspiration, piezoelectric pumping or the like.

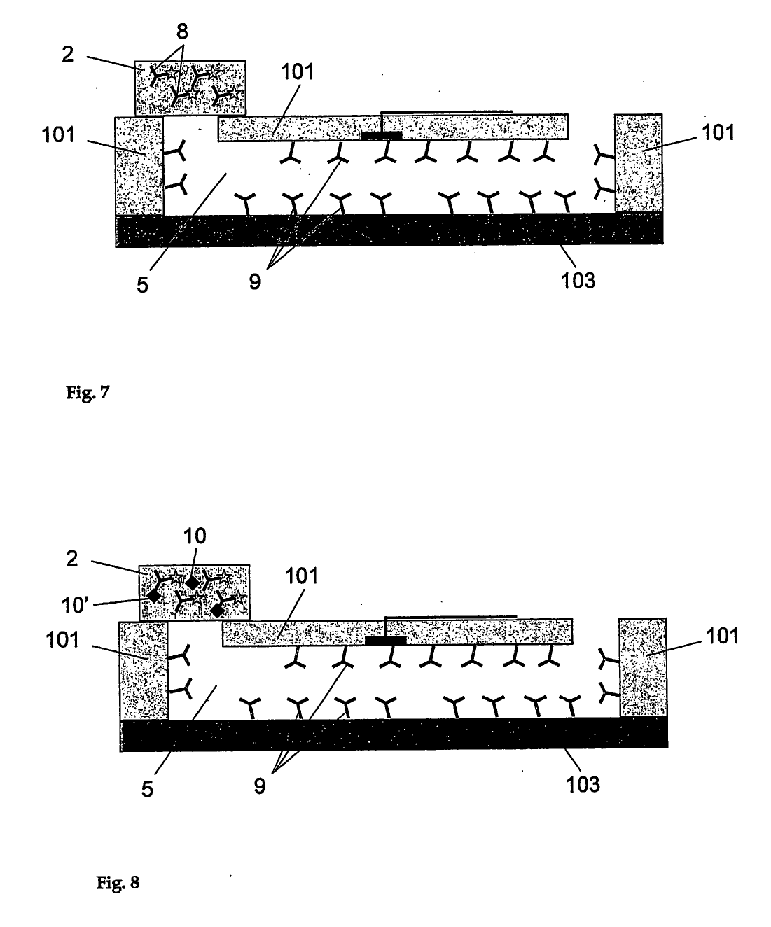

One object of this invention is indeed to provide a test strip specially designed for multi-reagent affinity assays (FIGS. 7 and 8). These test strips are composed of a porous layer 2 such as a cellulose or a PVDF membrane that contains a reagent such as e.g. a conjugate 8 (e.g. enzyme+DNA or enzyme+antibody), preferably in a dried form. The porous layer is placed on one entrance 5′ of a microstructure 5 (generally one or a network of covered microchannels) coated with a capture affinity molecule 9 (e.g. DNA or antibody), dose to and/or on a detection portion 4. The capture antibodies constitute a second reagent which, in this example, is immobilized directly on the walls of the fluidic connection and on the detection portion of the apparatus.

The detection portion 4 comprises, for example, an electrode, an ion selective membrane, an optical window, a waveguide or a nanospray end, and enables a qualitative and/or quantitative detection of an analyte in a test solution.

In a preferred embodiment, shown in FIG. 8, the test strip is dried and the test solution containing the analyte 10 (e.g. an antigen) is placed in contact with the porous layer (e.g. a BSA coated PVDF membrane) containing a conjugate that is solubilized upon addition of the test solution and that further reacts with the analyte 10 to form a product 10′ which is further transported to the detection portion of the apparatus placed in a microstructure 5 (e.g. a covered microchannel) coated with capture antibodies 9. The conjugate (which is labeled here with an enzyme) interacts in the porous layer to form a product 10′ which is here an antigen-conjugate complex.

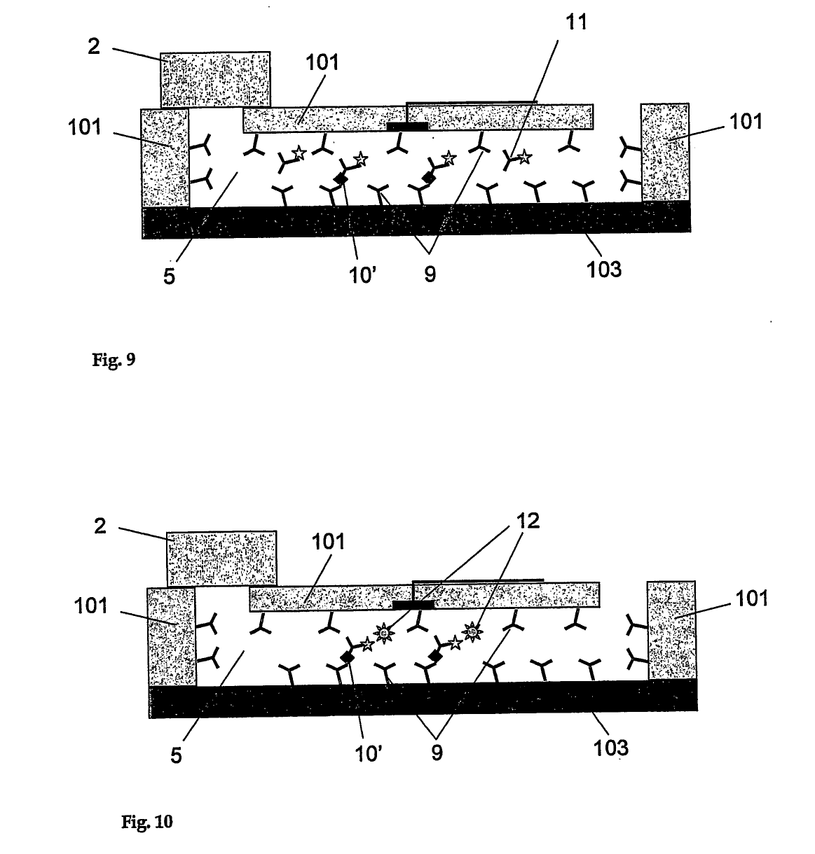

The antigen-conjugate complex will be captured by the capture antibodies inside the covered microchannel. As shown in FIG. 9, the solution present close to the detection portion comprises bound and unbound molecules, i.e. antigen-conjugate complexes 10′ that are bound or unbound to the antibody, as well as excess conjugate 11 that cannot bind to the antibody. A washing solution can be pumped into the microstructure in order to remove the non-bound species 11. FIG. 10 shows a substrate molecule 12 which can be added to the test solution or which can be introduced in the microstructure after passage of the test solution and possible washing steps. This substrate molecule is then transformed, e.g. by enzymatic reaction, into a product that can be detected at the detection portion (e.g. by reduction or oxidation on an electrode or by luminescence, etc).

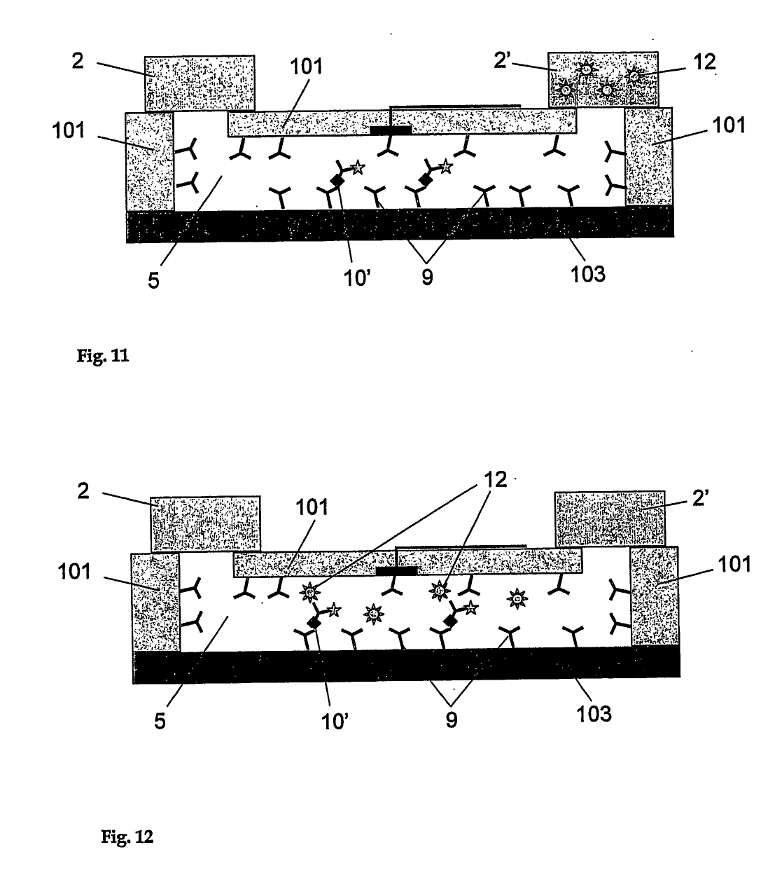

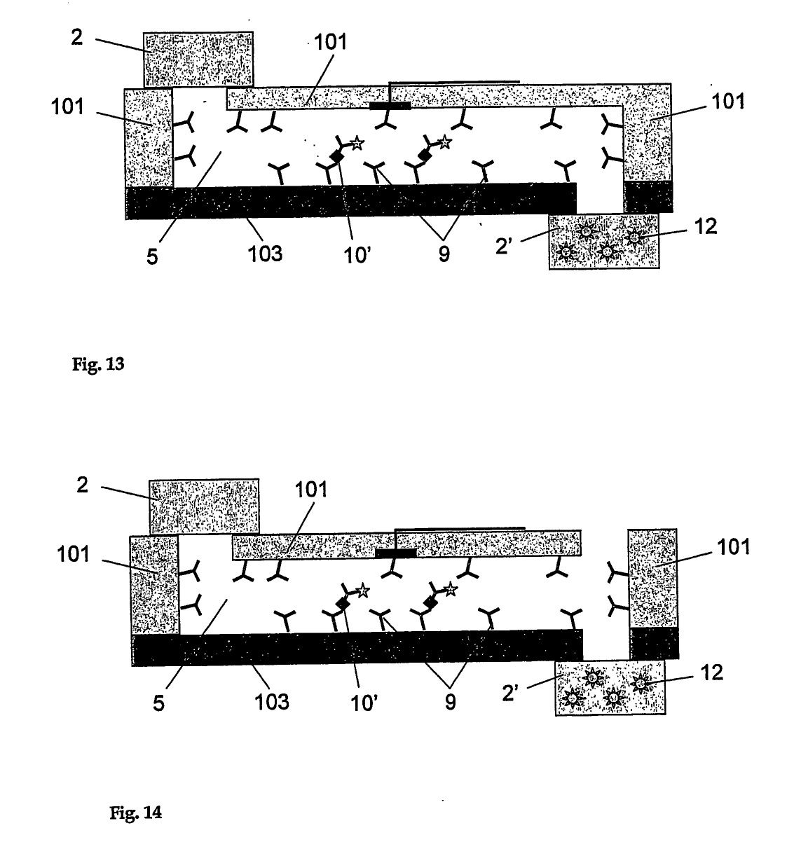

In another embodiment, the microchannel comprises a supplementary access hole with a second porous layer containing a dried substrate 12 (FIG. 11). A buffer solution can then be applied to this second porous layer so as to dissolve the supported substrate and introduce it into the microchannel (FIGS. 12 and 13) where this substrate is transformed into the product that is measurable at the detection portion of the microstructure. Optionally, no washing step is necessary between the immunological reaction and the delivery of the buffer through the second porous layer.

In some cases, the apparatus of this invention may comprise more than two access holes (inlet and outlet) to enter the detection portion such as to enable the adjunction of different reagents through a porous layer or directly by means of an external pumping system. In FIG. 14 two access holes are covered with a porous layer, and a third one is free to be connected to an external fluidic apparatus or to serve as a venting means.

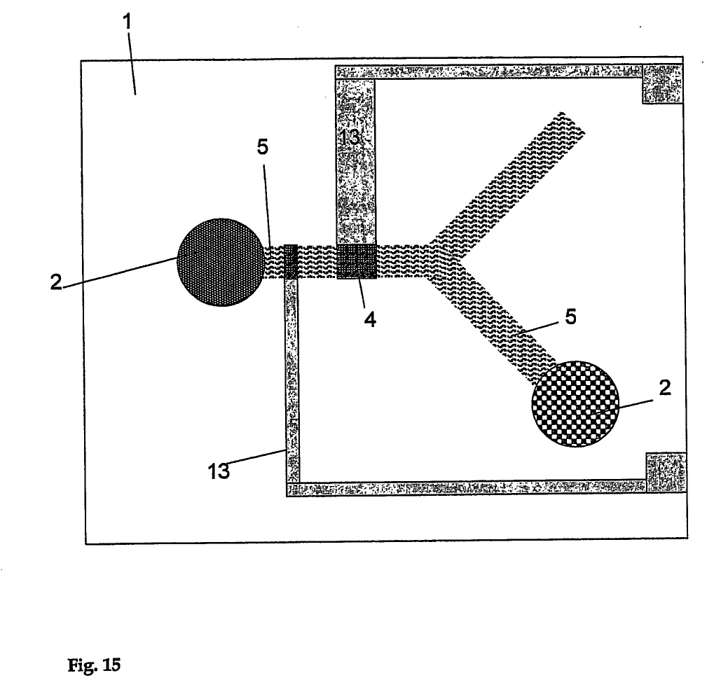

FIG. 15 is a top view of a test strip with a fluidic connection comprising a covered microchannel network having a Y-shape and exhibiting porous layers 2 connected to a detection portion being in this example integrated electrodes 4, said electrodes being connected to an external electrochemical unit with conductive tracks or waveguides (13).

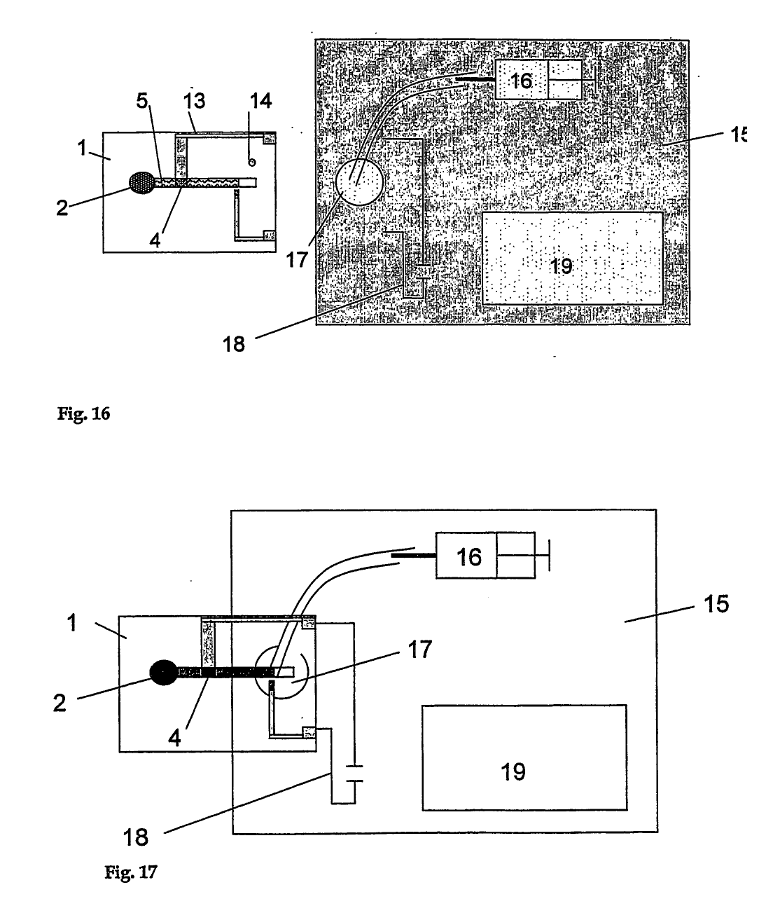

Various other features can be present in these microfluidic test strips such as conductive tracks 13 for connecting electrodes integrated in the detection portion, as well as registration holes or features 14 enabling clipping of the test strip in reading equipment 15 as shown in FIGS. 16 and 17. The detection portion of the test strip is connected to the reading equipment via a metallic contact 18 and the conductive tracks 13. The fluidic connection may be connected with a fluidic interface 17 for fluid introduction/uptake and/or control. The reading equipment enables the performance of fluidic control within the test strip, including aspiration and delivery of reagent using a solution reservoir 16, detection and optionally conversion of the detected signal into a comprehensive interpretation delivered on a screen 19. The information may either be stored in the apparatus or sent to a central information center by different telecommunication means, including e-mail, SMS, fax, telephone or the like. This assembly may serve as a systematic monitoring equipment in order to enter into theranostic systems such as the adaptation of a drug treatment to a diagnosis with the apparatus.

In order to easily connect the test strip to the apparatus as shown in FIG. 17, registration holes 14 in the test strip and corresponding features in the reading equipment may be present.

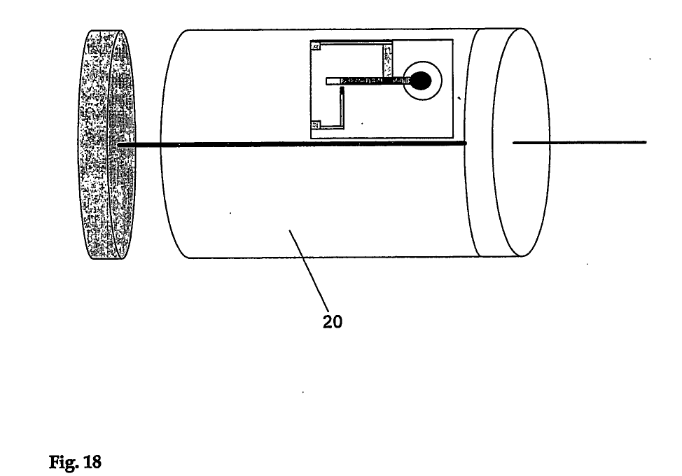

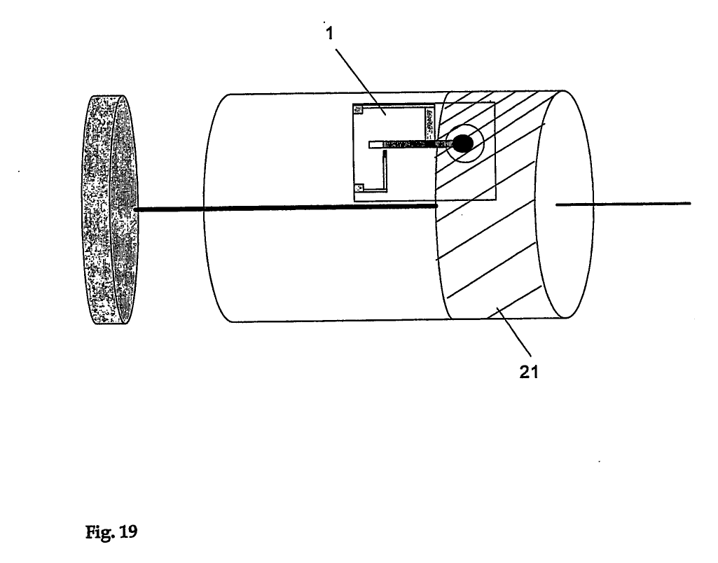

In another embodiment, the test strip may be incorporated to any device in contact with a body fluid so as to extract a sample and perform an analysis at any time and place. In some embodiments (FIGS. 18 and 19), the test strip is in contact with the external wall of a modified syringe 20 and a hole in the syringe enables blood 21 or another body fluid to be in contact with the porous layer of the test strip during a sample extraction. The syringe can be placed in contact with a reading device 15 so as to perform the final reading as already described above. The device containing the strip may be a storage bag, tubing or other pipette tips or catheter in contact with body fluids.

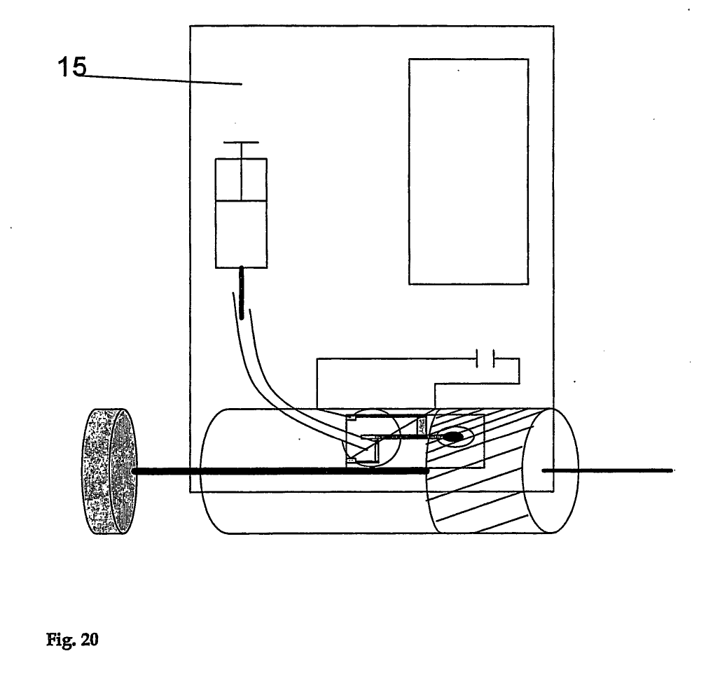

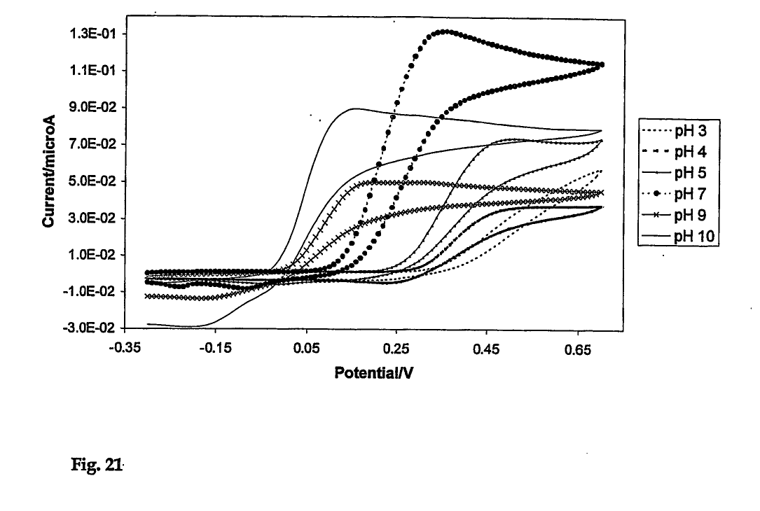

FIG. 20 is a view of the modified syringe connected to the reading device 15 so as to perform a multi-reagent test (for instance an immunological or an enzymatic assay) and to generate a comprehensive signal. Optionally, only the test strip can be taken and placed in contact with a reading equipment, similarly to the scheme of FIG. 17. In some cases, the apparatus can be integrated in the syringe itself.

Demonstration of the Invention

### EXAMPLE 1

### pH Measurement

In a first embodiment, the analyte to be tested in different test solutions is the proton concentration, namely the pH of the solution. In the apparatus shown in FIGS. 1 to 6, aminophenol (1 mM) is used as a reagent dried in the porous layer which is a cellulose membrane. This membrane is placed at the entrance of a microstructure serving as a fluidic connection. This microstructure is a 60 micrometer deep microchannel made of a 75 micrometer polyimide layer covered by a ˜40 micrometer thick polyethylene/polyethyleneterephthalate layer. The detection portion consists in an array of two microelectrodes that have an approximate diameter of 50 micrometers and that are made of copper coated by electroplated gold. These microelectrodes are part of the microchannel wall and exhibit a recess of about 15 micrometers. These microelectrodes are connected to an external potentiostat by way of gold/copper electrical tracks that are connected to a portable potentiostat (Palm Instruments, NL) enabling electrochemical measurements.

When the test solution reaches the membrane, the reagent is dissolved and driven into the detection portion. Due to the electrodes present in the microchannel and to a silver/silver chloride (Ag/AgCl) electrode in contact with the membrane, cyclic voltammetry of the solution is performed to oxidize the aminophenol in the detection portion of the apparatus. The oxidation potential of aminophenol depends on the concentration of protons present in solution and, therefore, the oxidation wave appears at different potentials corresponding to the different pH values of the test solutions as presented in FIG. 21.

It can be observed here that when the pH of the solution changes, the oxidation potential is shifted from approx 300 to −50 mV vs Ag/AgCl.

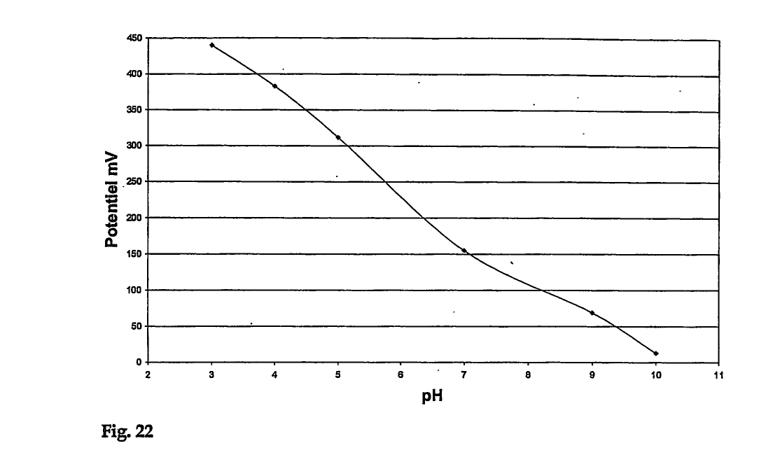

A calibration of the pH of the test solution can be undertaken as presented in FIG. 22. The role of the membrane is to provide the reagent as well as to retain undesired species such as particles, cells or large proteins.

### EXAMPLE 2

### Enzymatic Reaction (Case I: Substrate or Mediator in the Porous Layer)

In a further embodiment, the reagent placed in the porous layer is hydroquinone (HQ) which acts as a mediator in the enzymatic detection of the enzyme horseradish peroxidase (HRP) in the presence of H₂O₂ following the mechanism already described elsewhere Rossier et al. Lab-on-a-Chip, 2001,1,153-157).

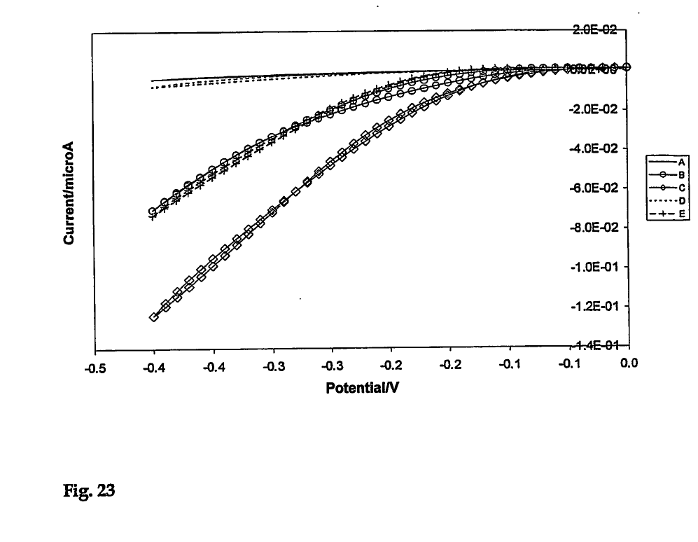

The following conditions are shown in FIG. 23:
A) cyclic voltammogram of the mixture of 10 mM hydroquinone and 10 mM H₂O₂ in phosphate buffer saline solution PBS pH 7.2;
B) cyclic voltammogram of the mixture of 10 mM hydroquinone and 10 mM H₂O₂ in phosphate buffer saline solution PBS pH 7.2 with HRP added to the solution;
C) cyclic voltammogram obtained with the apparatus shown in FIGS. 1 to 6 where hydroquinone is immobilized in the membrane as reagent and where H₂O₂ and horseradish peroxidase (HRP) are used as test solution in phosphate buffer saline solution PBS pH 7.2;
D) blank cyclic voltammogram obtained with the apparatus of FIGS. 1 to 6 when no hydroquinone is immobilized in the membrane (i.e. under absence of reagent) and where H₂O₂ and HRP are used as test solution in phosphate buffer saline solution PBS pH 7.2;
E) cyclic voltammogram obtained with the apparatus of FIGS. 1 to 6 where HRP is immobilized in the membrane as reagent, the membrane being blocked with 5% BSA and where H₂O₂ and hydroquinone are used as test solution in phosphate buffer saline solution PBS pH 7.2.

If HRP, HQ and H₂O₂ are mixed together and placed in a microchannel a reduction current can be detected as shown in FIG. 23 line B; this can be controlled so that with only the presence of HQ and H₂O₂ the cathodic current is much smaller as shown in line A. When a test solution containing HRP and peroxidase reaches the membrane (porous layer) containing HQ, the reagent (HQ) is dissolved and brought to the detection portion where an electrochemical detection can be performed as exemplified with detection line C.

In the presence of enzyme a reduction wave of Benzoquinone can be observed which is not the case in absence of the HQ in the membrane, done as a control experiment and shown as line D in FIG. 23.

### EXAMPLE 3

### Enzymatic Reaction (Case II: Enzyme in the Porous Layer)

In another embodiment, the enzyme is immobilized in the membrane so as to be dissolved by the test solution, here HQ/H₂O₂. The reaction occurs and the solution with the enzyme is brought towards the detection portion as shown in FIG. 23 line E where HRP was first dried in the membrane. In order to favor the dissolution and displacement of the enzyme towards the detection portion, the membrane can be precoated with bovine serum albumin (BSA) in order to avoid significant non specific adsorption of the enzyme in the membrane.

### EXAMPLE 4

### Affinity Assay

In this example the detection portion is coated with an affinity molecule such as a protein or a DNA strain. Avidine can be coated on the surface of the microchannel, and a biotinylated DNA capture probe is adsorbed on the avidine. The reagent in the porous layer is a DNA-HRP conjugate as described elsewhere (Rossier J S et al., CMEManager, 2002, 3, 28-32). The full test strip is dry and the test solution is placed on the porous layer. The analyte present in the test solution is a DNA strain complementary to the DNA-HPR conjugate present in the porous layer. Both analyte and reagent will complex and will be driven towards the detection portion inside the microchannel. There, the capture probe will react with the analyte and capture the complex. Using a pump connected to the microstructure on a free inlet, a washing solution can be flushed through the microstructure and above the detection portion of the apparatus so as to remove the non-immobilized DNA probe. Finally, a solution of 10 mM HRP and 10 mM H₂O₂ is brought to the detection portion where an enzymatic reaction will occur and can be detected by the reduction of the BQ mediator.

In another embodiment, the substrate mediator solution can be brought from another porous layer by passing buffer through the porous layer and directing the flow towards the detection portion.

1. A microfluidic assay apparatus for the detection of an analyte in a test solution, said microfluidic assay apparatus being a multi-layer body comprising:
a) a first, polymer layer, said polymer layer having at least one fluidic connection comprising a microstructure with at least one electrode integrated at a given location along said microstructure and forming a detection portion, said microstructure further comprising at least one inlet and one outlet and said at least one integrated electrode being connected to an external electrochemical unit;
b) a second, non-porous layer covering the microstructure so as to provide a sealed microstructure enabling microfluidic manipulations of a solution through said microstructure;
c) a third, porous layer, selected from a membrane, a glass frit, a sol-gel or a combination thereof and placed in contact with said first polymer layer at the inlet and/or outlet of said microstructure, said third porous layer comprising an immobilized reagent, said reagent interacting with said test solution to form a product that is transported through the porous layer and is released out of the porous layer inside the microstructure before being further transported inside said microstructure, so as to enable the detection of said analyte by way of said at least one integrated electrode.

2. An apparatus according to claim 1, wherein said reagent is reversibly immobilized in said third porous layer, said reagent being solubilized upon contact with said test solution and reacting with said analyte present in said test solution to form said product.

3. An apparatus according to claim 1, wherein said reagent is irreversibly immobilized in said third porous layer, said reagent enabling the capture of an undesirable compound or class of compounds present in the test solution or a given quantity of an analyte present in said test solution, said analyte or, respectively, the excess quantity of said analyte being transported through the porous layer and released out of the porous layer inside the microstructure before being further transported inside said microstructure, so as to enable the detection of said analyte by way of said at least one integrated electrode.

4-36. (canceled)

37. An apparatus according to claim 1, wherein said analyte is detected by electrochemistry using said at least one integrated electrode.

38. An apparatus according to claim 37, wherein said analyte is detected by way of an electron transfer reaction taking place at said at least one integrated electrode.

39. An apparatus according to claim 1, wherein said detection portion further comprises a selective ion-permeable membrane, an optical window, a waveguide, an electrospray tip and/or a piezoelectric means.

40. An apparatus according to claim 1, wherein said reagent immobilized in said third, porous layer is an antibody, an antigen, an enzyme, an oligonucleotide, DNA, RNA, a receptor, a cell, a peptide, a protein, or a ligand.

41. An apparatus according to claim 1, wherein said third, porous layer is sufficiently dense to retain particles including precipitates and blood cells.

42. An apparatus according to claim 1, where said microstructure is a microhole or microhole array, a millimeter hole, a covered microchannel or a covered microchannel array, a network of interconnected covered microchannels, or a gap between two plates.

43. An apparatus according to claim 1, wherein said microstructure is fabricated using photoablation, plasma etching, injection molding, embossing, casting, or silicone technology.

44. An apparatus according to claim 1, wherein said first, polymer layer and said second, non-porous layer are cut, glued, stacked, bonded, pressed or laminated together, so as to provide said microstructure and said detection portion.

45. An apparatus according to claim 1, wherein said second non-porous layer comprises a microstructure, an electrode and/or electrically conductive tracks.

46. An apparatus according to claim 1, wherein said external electrochemical unit is a potentiostat, a power supply or an impedance measurement system.

47. An apparatus according to claim 1, wherein said external electrochemical unit is adapted to measure and/or read a potential and/or a current.

48. An apparatus according to claim 1, wherein pumping means, pressure means and/or aspiration means are connected to said microstructure so as to uptake, deliver or withdraw a solution and/or control the flow of said solution in said fluidic connection.

49. An apparatus according to claim 1, wherein said detection portion is connected to said external electrochemical unit via electrically conductive tracks or optically conductive waveguides.

50. An apparatus according to claim 1, wherein at least one portion of said microstructure is filled with a medium, said medium being a solid, a gel or a sol-gel, a porous membrane, a monolithic column, beads or packed beads.

51. An apparatus according to claim 50, wherein said medium contains a reagent.

52. An apparatus according to claim 51, wherein said reagent is an antibody, an antigen, an enzyme, an oligonucleotide, DNA, RNA, a receptor, a cell, a peptide, a protein, or a ligand.

53. An apparatus according to claim 51, wherein said reagent is dried or immobilized in or on said medium.

54. An apparatus according to claim 53, wherein said reagent is immobilized by physisorption or covalent binding.

55. An apparatus according to claim 1, wherein at least one portion of said microstructure contains a reagent.

56. An apparatus according to claim 55, wherein said reagent is an antibody, an antigen, an enzyme, an oligonucleotide, DNA, RNA, a receptor, a cell, a peptide, a protein, or a ligand.

57. An apparatus according to claim 55, wherein said reagent is dried or immobilized on walls of said at least one portion of said microstructure.

58. An apparatus according to claim 57, wherein said reagent is immobilized by physisorption or covalent binding.

59. An apparatus according to claim 1, wherein said first, polymer layer forms a recess over said at least one integrated electrode.

60. An apparatus according to claim 1, wherein said apparatus is in contact with a modified syringe or tube or vessel or a patch, providing said test solution.

61. A method of performing a microfluidic assay comprising the steps of providing an apparatus according to claim 1, supplying said test solution to said porous layer, and detecting said analyte in said detection portion by way of said at least one integrated electrode.

62. A method according to claim 61, wherein said at least one integrated electrode is adapted to detect said analyte by electrochemistry.

63. A method according to claim 61, wherein said assay is selected from a pH measurement, a physico-chemical test, a biological assays, an ion, metal, enzyme, affinity, immunological, cellular, DNA, RNA haptamer, receptor, kinase or ligand assay.

64. A method according to claim 61, comprising a step of removing an interfering molecule or entity prior to the detection.

65. A method according to claim 61, wherein said microstructure is washed prior to detection of said analyte.

66. A method according to claim 61, wherein said apparatus is in contact with a modified syringe or tube or vessel or a patch, providing said test solution.

67. A method according to claim 66, wherein said apparatus is removed or replaced after analysis.

68. A method according to claim 61, wherein uptake, delivery, withdrawal or displacement of a solution or control of the flow of said solution in said microstructure is performed by pressure pumping, aspiration or electroosmosis.
